(19) United States
(12) Patent Application Publication
(10) Pub. No.: US 2014/0271491 A1
(43) Pub. Date: Sep. 18, 2014

(54) ADHESIVE MEDICAL PRODUCTS AND METHODS FOR TREATING GASTROINTESTINAL LESIONS

(71) Applicants: Shaun D. Gittard, Winston-Salem, NC (US); John Crowder Sigmon, JR., Winston-Salem, NC (US); Vihar C. Surti, Winston-Salem, NC (US)

(72) Inventors: Shaun D. Gittard, Winston-Salem, NC (US); John Crowder Sigmon, JR., Winston-Salem, NC (US); Vihar C. Surti, Winston-Salem, NC (US)

(73) Assignee: COOK MEDICAL TECHNOLOGIES LLC, Bloomington, IN (US)

(21) Appl. No.: 14/209,718

(22) Filed: Mar. 13, 2014

Related U.S. Application Data
(60) Provisional application No. 61/793,586, filed on Mar. 15, 2013.

(51) Int. Cl. A61L 26/00; A61L 15/24

(52) U.S. Cl. CPC A61L 26/0014; A61L 15/24
USPC 424/10.3; 424/78.06; 602/61

(57) ABSTRACT

The present invention relates to a long-lasting medical product for protecting or treating a lesion in the gastrointestinal tract. The medical product includes a protective covering, wherein the medical product upon application at and about the site of the lesion adheres to the gastrointestinal tissue and is capable of remaining at and about the site of the lesion for a time sufficient to allow the lesion to heal or be treated.

### RELATED APPLICATIONS

The present patent document claims the benefit of the filing date under 35 U.S.C. §119(e) of Provisional U.S. Patent Application Ser. No. 61/793,586, filed Mar. 15, 2013, which is hereby incorporated by reference.

### BACKGROUND

1. Technical Field

The present invention relates to adhesive medical products and methods for treating lesions in the gastrointestinal tract, such as lesions arising from the disorders of the gastrointestinal tract and or medical procedures that require removal of the mucosal or submucosal layers of gastrointestinal tract wall.

2. Background Information

There are several disorders of the gastrointestinal tract, e.g., gastrointestinal inflammation, gastrointestinal cancer, gastrointestinal infection, gastrointestinal motility dysfunction, or lesions, wounds or contusions of tissue of a portion of the gastrointestinal tract that can cause gastrointestinal lesions. In addition, there are a wide variety of medical procedures that require removal of the mucosal or submucosal layers of gastrointestinal tract wall and can also cause injury or lesions in the gastrointestinal tract. These procedures include endoscopic mucosal resection (EMR), endoscopic submucosal dissection, polypectomy, per-oral endoscopic myotomy, biopsy, and ablation (thermal, chemical, radiofrequency, and cryogenic). As with the disorders of the gastrointestinal tract, similar adverse events can occur after removal of the mucosal or submucosal layers, including bleeding and structuring.

Use of mucoadhesives and bioadhesives including various chemical derivatives of chitosan, Carbopol™ and polycarbophil has been known to open epithelial tight junctions, prevent intestinal ulceration, retain drugs in open wounds, increase ocular-surface residence, and have vaccine adjuvant activity, however, the use of mucoadhesives for the treatment of gastrointestinal lesion has not been previously proposed or documented.

Currently, the standard approach for treating gastrointestinal lesions is to use clips to fold the tissue upon itself, thus isolating the lesion from the gastrointestinal environment. However, this method has a poor success rate (10-20% failure), is expensive, and technically challenging.

As such, new or improved devices and methods for long-term localized treatment to address the issues arising from the disorders of the gastrointestinal tract and or medical procedures that require removal of the mucosal or submucosal layers of gastrointestinal tract wall are highly desirable.

### SUMMARY

In one embodiment, of the present invention relates to a long-lasting medical product for protecting or treating a lesion in the gastrointestinal tract comprising a protective covering, wherein the medical product upon application at and about the site of the lesion adheres to the gastrointestinal tissue and is capable of remaining at and about the site of the lesion for a minimum of 30 minutes. The protective covering may include an adhesive agent. The adhesive agent may be in a powder, a liquid or a gel form. The adhesive agent may be a mucoadhesive agent selected from carbomers (e.g., polyacrylic acids), polycyclic aromatic hydrocarbons (e.g., retene), carboxylic acids, polyvinylpyroolidones, polyvinylalchohols, polycarbophils, chitosan materials (e.g., poliglusam, deacetylchitin, or poly-(D)glucosamine), sodium alginates, cellulose derivatives (e.g., methylcellulose, methylethylcellulose, sodium carboxymethylcellulose, hydroxypropylcellulose, hydroxypropylmethylcellulose, or hydroxyethylcellulose), ethers (e.g., polyethylene glycol), lectins (e.g., Erythrina c. lectin, Concanavalin a. lectin, Ulex europaeus lectin, and C-Type lectin), thiamines (e.g. thiamine end capped polymer chains), pathogenic bacteria (e.g., bacterial fimbrins), thiols (e.g. chitosan-cysteine, chitosan-thiolbutylamidine, chitosan-thioglycolic acid, polyacrylic acid-cysteine, polyacrylic acid-cysteamine, carboxymethylellulose-cystein, or alginate-cysteine), amino acid sequences, ion-exchange resins (e.g., cholestyramine), any biomolecules including an amino acid sequence (e.g. peptides), mucin, guar gum, karya gum, xantham gum, locust bean gum, acacia gum, gellan gum, tragacanth, soluble starch, gelatin, pectin, and any biomolecules having an affinity for a mucosa (e.g., proteins, e.g., fimbrial proteins or affinity ligands). The medical product may also include a solvent, such as ethyl acetate, ethyl alcohol, water, DMSO, saline, acetone, isopropyl alcohol, or a combination thereof. The protective covering may also include a solid material in a form of a bandage, a patch, or a dressing. The medical product may be adapted for delivery in a liquid form that solidifies upon the delivery to and about the site of the lesion. The medical product may further include a mechanical scaffold. The mechanical scaffold can include an extracellular matrix or a synthetic material. The mechanical scaffold may include an impermeable material. In certain embodiments, the medical product may also include a color indicator. The medical product may be for protecting or treating gastrointestinal lesions resulting from endoscopic submucosal dissection, endoscopic mucosal resection, polypectomy, ulcers, cancers, varices, Barrett's esophagus ablation, infection, anastomoses, fistulas or a combination thereof. Preferably, the protective covering is capable of remaining at and about the site of the lesion for a minimum of 24 hours; more preferably for a minimum of 72 hours.

In another embodiment, the present invention relates to a method for protecting or treating a lesion in the gastrointestinal tract. The method includes locally applying a medical product comprising a protective covering to and about a lesion site in the gastrointestinal tract, wherein the medical product upon application at and about the site of the lesion adheres to the gastrointestinal tissue and is capable of remaining at and about the site of the lesion for a minimum of 30 minutes. In the method, the applying step includes placing the medical product at and about the site of the lesion. In the method, the applying step may include inserting a syringe loaded with the medical product about the site of the lesion and applying the medical product at and about the site of the lesion. In the method, the applying step may include spraying, ejecting or spreading the medical product at and about the site of the lesion. In the method, the applying step may be through endoscopic techniques, laparoscopic techniques, or direct access. In certain embodiments, the present method may further include applying a crosslinking initiator selected from the group consisting of thermal, light, curing agent or a catalyst. The method may further include instructing a medical practitioner to apply the medical product to and about a lesion site in the gastrointestinal tract.

In yet another embodiment, the present invention relates to a medical product for closing a perforation, anastomosis, or fistula of the gastrointestinal tract, the medical product including a protecting covering, wherein upon the application of the medical product, the protective covering forms a seal over the perforation, anastomosis, or fistula.

### BRIEF DESCRIPTION OF THE DRAWINGS

FIG. 1 is a photograph showing an example of a mucoadhesive covering of the present invention.

FIG. 2 is a photograph showing an example of a power form of the mucoadhesive covering on a post-mucosectomy site at time 0.

FIGS. 3A-D are photographs of tissue from the treated (A-B) and untreated (C-D) post-mucosectomy sites.

### DETAILED DESCRIPTION OF THE DRAWINGS AND THE PRESENTLY PREFERRED EMBODIMENTS

The present invention relates to long-lasting adhesive medical products and methods for protecting or treating lesions in the gastrointestinal tract resulting from disorders of the gastrointestinal tract and/or medical procedures that require removal of the mucosal or submucosal layers of gastrointestinal tract, such as, for example, endoscopic submucosal dissection, endoscopic mucosal resection, polypectomy, ulcer, cancer, varices, Barrett's esophagus ablation, or a combination thereof.

The long-lasting adhesive medical products of the present invention include a protective covering that may be, for example, a powder or a liquid coating, or a solid material, such as a bandage, a dressing, a patch or the like. The protective coating is delivered to and about a site of a lesion in the gastrointestinal tract to protect or treat the lesion from further injury or infection, slow or stop bleeding, prevent delayed bleeding, prevent delayed perforation, seal anastomotic leaks or fistulas, and/or promote healing at the exposed site.

In certain embodiments, the present invention relates to long-lasting adhesive medical products for protecting or treating lesions in the gastrointestinal tract.

The terms “protective coating” or “coating” or “protective covering” or “covering” encompass a powder and a liquid coating (e.g., solid, powder, liquid or gel suitable for applying, e.g., by spraying or coating or other known methods at and about the site of the lesion, where the liquid or gel coatings solidify at the site), as well as, a solid material, such as a bandage, a dressing, a patch and the like. Specifically, the terms encompass powder, liquid and gel coatings and solid materials (e.g., bandages, dressings, patches) for use in treating or protecting lesions in the gastrointestinal tract.

The term “lesion” refers to any tissue defect or bodily injury with disruption of the normal integrity of tissue structures and/or a pathological change in a bodily organ or tissue, and specifically, any bodily organ or tissue in the gastrointestinal tract. The term is also intended to encompass the terms “wound,” “injury,”“sore,” “necrosis” and “ulcer.” The term “sore” typically refers to any lesion of the mucous membranes. The term “ulcer” refers to a local defect, or excavation, of the surface of an organ or tissue in the gastrointestinal tract, which is produced by the sloughing of necrotic tissue. The term “necrosis” relates to dead tissue resulting from infection, injury, inflammation or infarctions. The lesion may be any lesion due to disorders of the gastrointestinal tract and or medical procedures that require removal of the mucosal or submucosal layers of gastrointestinal tract wall. For example, a lesion may be due to endoscopic submucosal dissection, endoscopic mucosal resection, polypectomy, ulcer, cancer, varices, Barrett's esophagus ablation, or a combination thereof. Specifically, upon a direct and localized delivery of the long-lasting adhesive medical product of the present invention to and about a site of a lesion, it will form a protective covering at and about the site of the lesion by at least partially or completely covering the lesion. The protective covering remains at and about the lesion site for a time sufficient to allow the site to be treated or healed (minimum of 30 minutes; preferably 24 hours; more preferably at least 48 or 72 hours; most preferably the protective covering is capable of remaining at and about the lesion site for 24-72 hours or longer; hence the term “long-lasting” refers to the time period that a protective covering of the present invention remains at and about the lesion and means anywhere from 30 minutes to 72 hours or longer). The term “protect” refers to protecting the site of the lesion from further injury or infection. The term “treat” refers to slowing or stopping bleeding at the site of the lesion, preventing delayed bleeding, preventing delayed perforation of the lesion, and/or promoting healing at the exposed site of the lesion, and/or promoting new tissue formation. The term “heal” in reference to a lesion refers to a process of repairing the gastrointestinal tissue by natural processes, as by, for example, scar formation so that following “healing” the lesion is at least reduced in size as compared to the initial size of the lesion or absent.

In certain embodiments, the long-lasting adhesive medical product of the present invention includes a protective covering that includes an adhesive agent.

An adhesive agent may include any currently known or future developed tissue adhesive agent(s). The adhesive may be a mucoadhesive or any other type of tissue adhesive. A mucoadhesive agent is preferred. As used herein, the terms “a mucoadhesive” or “a mucoadhesive agent” refer to an agent that adheres to a mucous membrane, which may line the wall of a body vessel or body cavity, e.g., a gastrointestinal surface (e.g., either or both of a gastrointestinal epithelia or mucosa (including submucosa) and, specifically, at and about a site of a lesion. The mucous membrane may include a moist mucous layer to which the mucoadhesive agent may adhere. Generally, mucoadhesive agents are hydrophilic macromolecules containing numerous functional groups capable of forming hydrogen bonds.

One example of a mucoadhesive agent suitable for use herein includes a macromolecule (e.g., a polymer) including repeating monomer units. Other examples of mucoadhesive agents for use in the long-lasting adhesive medical product of the present invention include, for example, a hydrophilic polymer, a hydrogel, a co-polymer, or a thiolated polymer. The hydrogen bond forming functional groups may include carboxyl groups, hydroxyl groups, carbonyl groups, sulphate groups, amide groups, or any other functional groups capable of forming hydrogen bonds. Examples of mucoadhesive agents or components thereof may include, for example, carbomers (e.g., polyacrylic acids), polycyclic aromatic hydrocarbons (e.g., retene), carboxylic acids, polyvinylpyroolidones, polyvinylalchohols, polycarbophils, chitosan materials (i.e., poliglusam, deacetylchitin, or poly-(D)glucosamine), sodium alginates, cellulose derivatives (e.g., methylcellulose, methylethylcellulose, sodium carboxymethylcellulose, hydroxypropylcellulose, hydroxypropylmethylcellulose, or hydroxyethylcellulose), ethers (e.g., polyethylene glycol), lectins (e.g., Erythrina c. lectin, Concanavalin a. lectin, Ulex europaeus lectin, and C-Type lectin), thiamines (e.g. thiamine end capped polymer chains); pathogenic bacteria (e.g., bacterial fimbrins), thiols (e.g. chitosan-cysteine, chitosan-thiolbutylamidine, chitosan-thioglycolic acid, polyacrylic acid-cysteine, polyacrylic acid-cysteamine, carboxymethylellulose-cystein, or alginate-cysteine), amino acid sequences, ion-exchange resins (e.g., cholestyramine), or any biomolecules including an amino acid sequence (e.g. peptides). Additional examples of mucoadhesive agents or components thereof may include mucin, guar gum, karya gum, xantham gum, locust bean gum, acacia gum, gellan gum, tragacanth, soluble starch, gelatin, or pectin. In some examples, mucoadhesive agents may include any biomolecules having an affinity for mucosal tissue such as, for example, proteins (e.g., fimbrial proteins or affinity ligands).

A mucoadhesive agent adheres to a mucous membrane by physical and/or chemical forces including, for example, ionic bonding, covalent bonding, hydrogen bonding, Van-der-Waals bonding, or hydrophobic bonding (i.e., hydrophobic interaction).

Other types of tissue adhesives include cyanoacrylate glues and sealants, glutaraldehyde, DOPA, or any other known polymer or biological adhesives.

In certain embodiments, medical products described herein as coverings comprise a mucoadhesive agent that may be in the form of a powder, a solid solution, an emulsion, a liquid or semi-liquid solution, a liquid suspension, a gel, a foam, or a combination thereof. Other known types of formulations may also be suitable for use and will be known to a skilled artisan.

The amount of the mucoadhesive in the product to be administered to and about the lesion will depend on the type and size of the lesion, the form of the mucoadhesive used and the delivery system used to deliver the mucoadhesive composition. For example, if the mucoadhesive is in the powder form, anywhere from a few tenths of a gram to several grams (e.g., anywhere from about 0.01 grams to about 25 grams) may be delivered and placed on and about the lesion site to provide a sufficient coverage at and about the lesion site, e.g., 10 microns to 2 mm thick. More typically, about 2 grams of the mucoadhesive powder would be sufficient to coat the lesion site (to achieve a thickness of anywhere from 10 microns to 2 mm thick). If the mucoadhesive is in the liquid form, enough mucoadhesive covering is provided to and about the lesion to allow sufficient coverage of the lesion, e.g., 10 microns to 2 mm thick.

The amount of the mucoadhesive in the liquid or gel mucoadhesive medical product of the present invention may vary from 0.1 to 99.5%.

In certain embodiments, any adhesive medical product or formulation described herein comprises greater than about 0.1% w/w, greater than about 0.2% w/w, greater than about 0.5% w/w, greater than about 1% w/w, greater than about 2% w/w, greater than about 3% w/w, greater than about 4% w/w, greater than about 5% w/w, greater than about 6% w/w, greater than about 7% w/w, greater than about 8% w/w, greater than about 9% w/w, greater than about 10% w/w, greater than about 11% w/w, greater than about 12% w/w, greater than about 13% w/w, greater than about 14% w/w, greater than about 15% w/w, greater than about 16% w/w, greater than about 17% w/w, greater than about 18% w/w, greater than about 19% w/w, greater than about 20% w/w, greater than about 21% w/w, greater than about 22% w/w, greater than about 23% w/w, greater than about 24% w/w, greater than about 25% w/w, greater than about 26% w/w, greater than about 27% w/w, greater than about 28% w/w, greater than about 29% w/w, greater than about 30% w/w, greater than about 35% w/w, greater than about 40% w/w, greater than about 45% w/w, greater than about 50% w/w, greater than about 55% w/w, greater than about 60% w/w, greater than about 65% w/w, greater than about 70% w/w, greater than about 80% w/w, greater than about 85% w/w, greater than about 90% w/w, greater than about 95% w/w, or greater than about 99.5% w/w of mucoadhesive that can extend the time the adhesive medical product is in contact with a surface of the gastrointestinal tract (e.g., the surface of the stomach).

As provided herein, medical products in a form of a powder may include finely divided or subdivided preparations, coarsely comminuted products, or products of intermediate particle size. A medical product in a form of a powder can comprise particles which are very coarse, of the dimensions of about 10,000 microns or 10 mm, or particles which are extremely fine, approaching dimensions of about 1 micron or less, or particles of any size in between coarse and fine. The size of the particles will depend on the type of the mucoadhesive used in the formulation and may be highly variable especially for the granular mucoadhesive.

In addition, powders can contain certain proportions of liquids dispersed thoroughly and uniformly over the solid components of the mixture, or can be composed entirely of solid materials.

In certain embodiments, the medical products may include one or more mucoadhesive agents. For example, a medical product in a form of a powder can be a physical admixture of two or more powdered pure mucoadhesive agents present in definite or differing proportions. For example, a medical product in a form of a liquid can be a physical admixture of two or more powdered pure mucoadhesive agents present in definite or differing proportions mixed with a solvent.

In some embodiments, therapeutic agents and or the excipients may be mixed in with the mucoadhesive powder into a powder protective covering formulation suitable for use according to the methods of this invention or be mixed in with the mucoadhesive liquid into a liquid protective covering formulation.

In an alternative embodiment, protective coverings in the form of powders or granules can be reconstituted with a solvent or water or other liquid before use. Upon reconstitution, the formulations in the form of powders or granules can be mixed with dyes, colorants, flavorants, and/or other desired pharmaceutical ingredients, so the reconstituted solution can have pharmaceutical features of a liquid pharmaceutical.

Powders used for the purpose of formulations provided herein include formulations where a physician can use the formulation as is (i.e., in a powder form) or mix a directed amount of powder (typically a teaspoon) with a directed amount of a solvent or water or other liquid followed by localized administration at and about the site of the lesion and is capable of remaining at and about the site of the lesion anywhere from 30 minutes (sufficient to stop bleeding) to 72 hours or more (sufficient to allow the tissue to heal almost completely) following the delivery.

In certain embodiments, the protective covering may include multiple materials.

In one embodiment, the protective covering may include, e.g., an adhesive material (to adhere to the tissue) and a super-absorbent material (to act as a barrier to prevent liquids from contacting the lesion or blood from leaving the lesion site). Additionally or alternatively, the protective covering may include a hemostatic material (to coagulate blood in the event that the lesion bleeds) as one of its components.

In yet another embodiment, an additive(s) to promote healing may be included in the protective covering.

Some examples of super-absorbent materials include absorbent clays (e.g., Laponite, Smectites, Zeolites), Diatomaceous Earth, and super-absorbent polymers (e.g., sodium polyacrylate, polyacrylamide copolymer, ethylene maleic anhydride copolymer, cross-linked carboxymethylcellulose, polyvinyl alcohol copolymers, cross-linked polyethylene oxide, and starch-acrylonitrile co-polymer).

Some examples of hemostatic materials include Alginate, Chitin, Chitosan, Collagen, Fibrin, Kaolinite clays, Oxidized Cellulose, Plant-based polysaccharides, Platelets, Smectite clays, and Zeolites.

Some examples of additives to promote healing include Aloe vera and derivatives, Honey and derivatives (i.e. Leptospermum scoparium honey), and Growth factors.

In certain embodiments, the medical product of the present invention may also include a mechanical scaffold to form a bandage, a patch, a dressing or the like. The term “scaffold” refers to any natural (e.g., extracellular matrix material or “ECM”), synthetic (e.g., woven or non-woven) material (e.g., Dacron™, electrospun materials and expanded PTFE) capable of providing a mechanical and structural support and strength to the protective covering of the adhesive medical product of the present invention.

The medical products of the present invention employ scaffolds which may be applied in a variety of forms, including single- or multi-layer sheet or mesh constructs, fluidized formulations, and/or combinations thereof.

Examples of synthetic scaffolds include biocompatible materials, such as polyesters, such as polyethylene; poly(ethylene terephthalate); fluorinated polymers, such as polytetrafluoroethylene (PTFE) and fibers of expanded PTFE; polyurethanes; silicone, etc. One example of biocompatible polyester includes Dacron™ (DuPONT, Wilmington, Del.).

Examples of natural scaffold materials include bioremodelable ECM-based materials, such as naturally-derived collagenous ECM materials isolated from suitable animal or human tissue sources. As used herein, it is within the definition of a “naturally-derived ECM” to clean, delaminate, and/or comminute the ECM, or to cross-link the collagen or other components within the ECM. It is also within the definition of naturally occurring ECM to fully or partially remove one or more components or subcomponents of the naturally occurring matrix. Bioremodelable ECM materials possess biotropic properties capable of inducing tissue remodeling. Suitable ECM materials which can be processed to provide scaffold materials include, for example, submucosal (including for example small intestinal submucosa (SIS), stomach submucosa, urinary bladder submucosa, or uterine submucosa, each of these isolated from juvenile or adult animals), renal capsule membrane, dermal collagen, amnion, dura mater, pericardium, serosa, peritoneum or basement membrane layers or materials, including liver basement membrane or epithelial basement membrane materials, and others.

An exemplary ECM sheet material is a sheet of submucosa tissue graft material (OASIS™ Wound Matrix, Cook Biotech Incorporated, West Lafayette, Ind., USA).

A medical product containing a scaffold and a mucoadhesive can be used in the treatment of a lesion in the gastrointestinal lesion. For these purposes, the scaffold material can be processed into the form of a sheet, bandage or other shape to occlude or cover at least partially the lesion site in the gastrointestinal tract.

In certain embodiments of the present invention, one or more adhesive (e.g., mucoadhesive) agents may be mixed with fluidized ECM material to form a substantially homogenous adhesive solution. The fluidized ECM material may be dried or formed into a gel for direct use.

In certain embodiments, comminuted submucosal or other ECM material can be dried by freeze drying to form a powder, which can hydrated, that is, combined with water or buffered saline and optionally other pharmaceutically acceptable excipients, to form a fluid ECM adhesive medical product. The viscosity of fluidized ECM compositions may be manipulated by controlling the concentration of the submucosa or other ECM components, the degree of hydration and adjusting the pH of the submucosal or other ECM digest. The viscosity may be adjusted to a range of about 2 to about 300,000 cps at 25° C. Higher viscosity gel formulations can have a gel or paste consistency and may be prepared by adjusting the pH of the digest solutions to about 6.0 to about 7.0.

Alternatively, the fluidized ECM material may be dried and adhered to or coated with the mucoadhesive agent(s).

In certain embodiments, the adhesive medical product of the present invention can be a composite medical product including additional layers such as biocompatible substrate films or layers. For example, the medical products may include a top sheet or impermeable layer to restrict passage of liquid, such as gastric acid back towards the lesion. Alternatively, or in addition, the medical product of the present invention may include an additional backing layer providing further barrier or structural support.

In certain embodiments, the adhesive may be incorporated into the scaffold by mixing, coating, spraying, impregnating the scaffold with the adhesive or may be provided as a separate adhesive layer forming an adhesive-coated margin.

In certain embodiments, the medical product of the present invention may include a mucoadhesive covering on the interface that is placed on the gastrointestinal tissue and an additional material such as a woven mesh scaffold on the lumen interface to provide improved mechanical strength to the medical product.

In certain embodiments, the protective covering of the long-lasting adhesive medical product disclosed herein and used herein may comprise excipients. Specifically, additional excipients useful herein include, by way of non-limiting example, solvents, binders, fillers, lubricants, suspension agents, flavoring agents, color indicators (e.g., dyes), sweeteners, preservatives, antioxidants, buffering agents, humectants, chelating agents, surfactants, disintegrating agents, and the like. These excipients can extend the time the adhesive medical product of the present invention is in contact with a lesion site in the gastrointestinal tract and/or can increase the interaction of the adhesive medical product with a gastrointestinal surface, such as viscosity enhancing agents or absorption enhancing agents may be used.

In certain embodiments, the protective covering of the present invention include a solvent. Exemplary solvents include ethyl acetate, ethyl alcohol, water, DMSO, saline, acetone, isopropyl alcohol, or a combination thereof. If a solvent is used, the resultant mucoadhesive product in the form of a liquid or a gel and may be delivered as such to the site of the lesion.

Optionally, the present protective covering may include one or more binder, optionally one or more filler, optionally one or more lubricant, optionally one or more suspension agent, optionally one or more flavoring agent, optionally one or more coloring agent, optionally one or more sweetener, optionally one or more preservative, optionally one or more antioxidant, optionally one or more buffering agent, optionally one or more humectant, optionally one or more chelating agent, optionally one or more disintegrating agent, and optionally one or more surfactant.

In addition, the protective covering of the present invention may include, for example, a color indicator, such as a dye that upon the application to and about the lesion site changes color. Water soluble dyes are preferable. Exemplary dyes include indigo carmine, methylene blue, fluorescent proteins, Rose Bengal, India ink, and others.

Preservatives include, for example, benzalkonium chloride, cetrimide (cetyltrimethylammonium bromide), benzoic acid, benzyl alcohol, methyl-, ethyl-, propyl- and butyl-esters of para-hydroxybenzoic acid, chlorhexidine, chlorobutanol, phenylmercuric acetate, borate and nitrate, potassium sorbate, sodium benzoate, sorbic acid, thiomersal (mercurithiosalicylate), combinations thereof, or the like.

Antioxidants include, for example, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, edetate (EDTA) (e.g., disodium edetate), Diethylenetriaminepentaacetic acid (DTPA), Triglycollamate (NT), combinations thereof, or the like.

Additionally, in certain embodiments, buffering agents, humectants, or chelating agents may also be incorporated into the protective covering of the adhesive medical products of the present invention.

Exemplary buffering agents include citrate buffers (i.e., citric acid and citrate), phosphate buffers, acetate buffers, carbonate buffers (e.g., calcium carbonate, sodium bicarbonate, or the like), hydroxide (e.g., magnesium hydroxide, sodium hydroxide, or the like), combinations thereof, or the like.

Humectants include, for example, glycerine, propylene glycol, ethylene glycol, glyceryl triacetate, polyols (e.g., sorbitol, xylitol, maltitol, polydextrose), and the like.

Chelating agents include, for example, edetate (EDTA) (e.g., disodium edetate), Diethylenetriaminepentaacetic acid (DTPA), Triglycollamate (NT), or the like.

In some embodiments, the adhesive medical products described herein are pharmaceutical medical products further including at least one therapeutic agent. Exemplary therapeutic agents that may be incorporated into the long-lasting adhesive medical products of the present invention include antibiotics, antiseptic agents, proton pump inhibitors, or tissue growth promoting compounds.

Other therapeutic agents may also be included and would be known to a skilled artisan.

In certain embodiments, the adhesive medical products of the present invention are prepared for a local and direct delivery at and about a site of a lesion, where the medical product upon the delivery or application to the lesion forms a coating or a patch or the like at and about the site of the lesion and is capable of remaining at and about the site of the lesion for a time sufficient to allow the site to be treated or healed. Preferably, at least partial coverage of the lesion site is achieved; most preferably a complete coverage of the lesion site is achieved. The time sufficient to allow the site to be treated or healed may vary depending on the location, type and size of the lesion and may be anywhere from 30 minutes (sufficient to stop bleeding) to 72 hours or more (sufficient to allow the tissue to heal almost completely). Preferably, the time sufficient to allow the site to be treated or healed is at least 1 hour; more preferably the time sufficient to allow the site to be treated or healed is at least 3 hours; more preferably the time sufficient to allow the site to be treated or healed is at least 6 hours; more preferably the time sufficient to allow the site to be treated or healed is at least 10 hours; more preferably the time sufficient to allow the site to be treated or healed is at least 12 hours; more preferably the time sufficient to allow the site to be treated or healed is at least 18 hours; more preferably the time sufficient to allow the site to be treated or healed is at least 24 hours; more preferably the time sufficient to allow the site to be treated or healed is at least 36 hours; more preferably the time sufficient to allow the site to be treated or healed is at least 48 hours; more preferably the time sufficient to allow the site to be treated or healed is at least 72 hours; most preferably the time sufficient to allow the site to be treated or healed is 48-72 hours.

### Delivery

In certain embodiments, the adhesive medical products of the present invention may be applied or delivered to and about the site of the lesion through endoscopic techniques, laparoscopic techniques, or through direct access (i.e., surgically) using, for example any suitable catheter delivery system.

In certain embodiments, the adhesive medical products of the present invention may be delivered via an intraluminal delivery system. The adhesive medical products of the present invention may be applied via spraying, ejecting, injecting or spreading.

In certain embodiments, where the adhesive medical product of the present invention includes a protective covering that includes multiple mucoadhesive agents, the delivery of the multiple mucoadhesive agents may be through a multi-lumen catheter. In certain embodiments, the multiple mucoadhesive agents delivered to and about the lesion site may be mixed following the delivery at and about to lesion site.

Specifically, the delivery system may include a delivery device that is sized and configured to deliver and apply the adhesive medical product of the present invention directly at a targeted tissue region within a body lumen or hollow body organ, i.e., such as the site of the lesion in the gastrointestinal tract.

Also, the delivery device can be sized and configured to accommodate passage over a guide wire. In this way, the device can be introduced over the guide wire under direct visualization from an endoscope. Specifically, the guide wire can run next to the endoscope and therefore leaves a working channel of the endoscope free. In an alternative embodiment, the delivery device can be sized and configured to be back-loaded through the working channel of the endoscope. The working channel of the endoscope thereby serves to guide the delivery device while providing direct visualization.

### Use

The medical products of the invention can find wide use in the field of medicine, and in this regard, can be adapted to provide a variety of devices and objects suitable for application to and/or implantation within a patient, and especially in the gastrointestinal tract. The present invention also provides, in certain aspects, various methods for using these materials, for example, to replace, augment, repair, and/or otherwise suitably treat diseased or otherwise damaged or defective gastrointestinal tissue of a patient.

Illustratively, medical products of the invention can be configured as medical products suitable for healing tissue, providing hemostasis, and/or providing occlusion within the body of a patient (e.g., bandage, dressing, patch, etc.).

In some embodiments, the adhesive medical products of the present invention are configured as single- or multilayered patches or other sheet or sheet-like devices for providing support to patient tissue or otherwise treating patient's gastrointestinal tissue.

Specifically, the long-lasting adhesive medical products of the present invention may be used to protect, treat or heal a lesion site in the gastrointestinal tract. Specifically, the present adhesive medical products may be used to treat a lesion arising from the disorders of the gastrointestinal tract and or medical procedures that require removal of the mucosal or submucosal layers of gastrointestinal tract wall, such as endoscopic submucosal dissection, endoscopic mucosal resection, polypectomy, ulcer, cancer, varices, Barrett's esophagus ablation, a combination thereof, or others.

Illustratively, medical products of the present invention can be processed into various shapes and configurations, for example, a sheet form, which may be used as a bandage, patch, coating, etc.

In additional embodiments, the medical product of the present invention may be used for closing a perforation, anastomosis, or fistula of the gastrointestinal tract. The medical product comprises a protecting covering as discussed above, wherein upon the application of the medical product, the protective covering forms a seal over the perforation, anastomosis, or fistula. The medical product may be used in combination with other medical products, such as clips or sutures.

### Methods

In another embodiment, the present invention is directed to a method for protecting or treating a lesion in the gastrointestinal tract arising from the disorders of the gastrointestinal tract and or medical procedures that require removal of the mucosal or submucosal layers of gastrointestinal tract wall. The lesion may be a post-mucosectomy lesion.

The method includes locally applying an adhesive medical product that includes a protective covering to and about the lesion in the gastrointestinal tract. The phrases “to and about” or “at and about” in connection with the delivery of the long-lasting adhesive medical products of the present invention mean that the adhesive medical product is placed on the lesion itself as well as just immediately around the lesion to ensure as most complete coverage of the lesion as possible. Upon the application of the medical product, the product forms a protective coating or covering at the site of the lesion, where the coating is capable of remaining at and about the site of the lesion for at least 30 minutes, more preferably 24-72 hours or longer.

In certain embodiments, the composition may be applied in a powder form. Alternatively, the composition may be applied in a liquid or gel form. The types of mucoadhesive medical products and formulations suitable for use in the methods of this invention were described in detail above.

In certain embodiments, the step of applying the adhesive medical product comprises inserting a syringe loaded with the protective covering about the site of the lesion and applying the covering at and about the site of the lesion by spraying, injecting, ejecting or spreading the composition directly at and about the site of the lesion so as to provide at least partial but more preferably a complete coverage of the lesion. As previously discussed in connection with the delivery methods, the applying may be through endoscopic, laparoscopic techniques or direct access (i.e., surgically) using a catheter-based delivery system (single lumen or multi-lumen catheter system).

In certain embodiments, a crosslinking initiator, such as thermal, light, curing agent or a catalyst may be used to aid in the process of solidifying of the coating at and about the lesion site.

Without being bound by the theory, upon the application of the adhesive medical product or protective covering of the present invention to the lesion site, the covering will remain at and about the site of the lesion for the time sufficient for the lesion to heal (anywhere from 30 minutes to 72 hours or longer). Once the lesion is healed, the covering will either get washed off or erode over time; the covering will then be passed through the digestive system for removal from the body. Alternatively, the covering will remain at the site of the lesion until the outer most mucosal layer sloughs off of sheds during a normal biological process over 2-3 weeks and the coating or covering comes off with the mucosal layer.

The following examples are included to demonstrate certain embodiments of the invention. Those of skill in the art should, however, in light of the present disclosure, appreciate that modifications can be made in the specific embodiments that are disclosed and still obtain a like or similar result without departing from the spirit and scope of the invention. Therefore, the examples are to be interpreted as illustrative and not in a limiting sense.

### EXAMPLES

### Example 1

### Liquid Mucoadhesive Benchtop Testing

Various solutions of Carbopol™ were tested: (1) Carbopol™ dissolved in ethyl acetate, (2) Carbopol™ dissolved in ethyl alcohol, and (3) Carbopol™ dissolved in water. The Carbopol™ solutions were applied to excised stomach and intestinal tissue.

Specifically, all three solutions of Carbopol™ were loaded into a syringe and injected directly at the lesion site through a catheter. Ethyl acetate (as shown in FIG. 1) was found to be a most suitable solvent to allow the highest concentration of Carbopol™. The next best was ethyl alcohol followed by water which was necessary to be highly dilute.

Next, the dye indigo carmine was mixed with the ethyl alcohol solvent. This solution remained white, with small blue particles of the dye suspended in it. However, upon application to the tissue the suspension turned blue as water from the tissue became absorbed into the adhesive. This suggests that use of a dye may be suitable for visualization of the lesion site that is being treated.

### Example 2

### Animal Survival Testing

To determine the sustainability and protective effects of a coating over an extended period of time (up to 72 hours), a mucoadhesive coating was applied to post-mucosectomy sites and compared to untreated (negative control) mucosectomy sites.

Specifically, to test the sustainability of Carbopol™ 71G NF powder, the powder was sprayed onto 5 post-mucosectomy sites in a live animal and compared to five negative controls (mucosectomy sites without the application of the spray) at 72 hours post treatment. At 72 hours following the application of the Carbopol™ 71G NG powder, the animal was sacrificed and tissue samples were harvested for gross and histological examinations.

FIG. 2 is an example of a power form of the mucoadhesive coating on a post-mucosectomy site at time 0.

Referring to FIGS. 3A-D, an initial gross examination revealed a thin gel layer still residing over the Carbopol™ powder-treated post-mucosectomy sites at 72 hours following the application of the Carbopol™ powder (FIG. 3A-B). Also, the Carbopol™ powder-treated post-mucosectomy sites (FIGS. 3A-B) looked significantly more healed as compared to the negative controls (FIGS. 3C-D) sites.

These results suggest that the application of a mucoadhesive coating to a post-mucosectomy site may have a protective effect and enhance healing of the injured site.

1. A long-lasting medical product for protecting or treating a lesion in the gastrointestinal tract comprising a protective covering, wherein the medical product upon application at and about the site of the lesion adheres to the gastrointestinal tissue and is capable of remaining at and about the site of the lesion for a minimum of 30 minutes.

2. The medical product of claim 1, wherein the protective covering comprises an adhesive agent.

3. The medical product of claim 2, wherein the adhesive agent is in a powder, a liquid or a gel form.

4. The medical product of claim 2, wherein the adhesive agent is a mucoadhesive agent.

5. The product of claim 4, wherein the mucoadhesive agent is selected from the group consisting of carbomers (e.g., polyacrylic acids), polycyclic aromatic hydrocarbons (e.g., retene), carboxylic acids, polyvinylpyroolidones, polyvinylalchohols, polycarbophils, chitosan materials (e.g., poliglusam, deacetylchitin, or poly-(D)glucosamine), sodium alginates, cellulose derivatives (e.g., methylcellulose, methylethylcellulose, sodium carboxymethylcellulose, hydroxypropylcellulose, hydroxypropylmethylcellulose, or hydroxyethylcellulose), ethers (e.g., polyethylene glycol), lectins (e.g., Erythrina c. lectin, Concanavalin a. lectin, Ulex europaeus lectin, and C-Type lectin), thiamines (e.g. thiamine end capped polymer chains), pathogenic bacteria (e.g., bacterial fimbrins), thiols (e.g. chitosan-cysteine, chitosan-thiolbutylamidine, chitosan-thioglycolic acid, polyacrylic acid-cysteine, polyacrylic acid-cysteamine, carboxymethylellulose-cystein, or alginate-cysteine), amino acid sequences, ion-exchange resins (e.g., cholestyramine), any biomolecules including an amino acid sequence (e.g. peptides), mucin, guar gum, karya gum, xantham gum, locust bean gum, acacia gum, gellan gum, tragacanth, soluble starch, gelatin, pectin, and any biomolecules having an affinity for a mucosa (e.g., proteins, e.g., fimbrial proteins or affinity ligands).

6. The product of claim 2, further comprising a solvent.

7. The medical product of claim 6, wherein the solvent is ethyl acetate, ethyl alcohol, water, DMSO, saline, acetone, isopropyl alcohol, or a combination thereof.

8. The medical product of claim 1, wherein the protective covering comprises a solid material.

9. The medical product of claim 8, wherein the solid material is in a form of a bandage, a patch, or a dressing.

10. The medical product of claim 1, further comprising a super-absorbent material, a hemostatic material, or an additive.

11. The medical product of claim 4, wherein the medical product is adapted for delivery in a liquid form that solidifies upon the delivery to and about the site of the lesion.

12. The medical product of claim 2, further comprising a mechanical scaffold.

13. The medical product of claim 12, wherein the mechanical scaffold comprises an extracellular matrix or a synthetic material.

14. The medical product of claim 12, wherein the mechanical scaffold comprises an impermeable material.

15. The medical product of claim 1, further comprising a color indicator.

16. The medical product of claim 1, wherein the product is for protecting or treating gastrointestinal lesions resulting from endoscopic submucosal dissection, endoscopic mucosal resection, polypectomy, ulcers, cancers, varices, Barrett's esophagus ablation, infection, anastomoses, fistulas or a combination thereof.

17. The medical product of claim 1, wherein the product is capable of remaining at and about the site of the lesion for a minimum of 24 hours.

18. The medical product of claim 1, wherein the product is capable of remaining at and about the site of the lesion for a minimum of 72 hours.

19. A method for protecting or treating a lesion in the gastrointestinal tract, comprising locally applying a medical product comprising a protective covering to and about a lesion site in the gastrointestinal tract;
wherein the medical product upon application at and about the site of the lesion adheres to the gastrointestinal tissue and is capable of remaining at and about the site of the lesion for a minimum of 30 minutes.

20. The method of claim 19, wherein the applying step comprises placing the medical product at and about the site of the lesion.

21. The method of claim 19, wherein the applying step comprises inserting a syringe loaded with the medical product about the site of the lesion and applying the medical product at and about the site of the lesion.

22. The method of claim 19, wherein the applying step comprises spraying, ejecting or spreading the medical product at and about the site of the lesion.

23. The method of claim 19, wherein the applying step is through endoscopic techniques, laparoscopic techniques, or direct access.

24. The method of claim 19, further comprising applying a crosslinking initiator selected from the group consisting of thermal, light, curing agent or a catalyst.

25. The method of claim 19, further comprising instructing a medical practitioner to apply the medical product to and about a lesion site in the gastrointestinal tract.

26. A medical product for closing a perforation, anastomosis, or fistula of the gastrointestinal tract, the medical product comprising a protecting covering, wherein upon the application of the medical product, the protective covering forms a seal over the perforation, anastomosis, or fistula.
